(19) United States
(12) Patent Application Publication
(10) Pub. No.: US 2009/0203950 A1
(43) Pub. Date: Aug. 13, 2009

(54) Metathesis process using a fluidized bed reactor

(76) Inventors: Richard B. Halsey, Houston, TX (US); Steven T. Coleman, Humble, TX (US)

Correspondence Address: LyondellBasell Industries, 3801 WEST CHESTER PIKE, NEWTOWN SQUARE, PA, 19073, US

(21) Appl. No.: 12/069,811

(22) Filed: Feb. 12, 2008

(51) Int. Cl. C07C 6/02

(52) U.S. Cl. 585/646

(57) ABSTRACT

A method for metathesizing at least two olefins using a fluidized bed metathesis reactor and a finely subdivided solid catalyst.

### BACKGROUND OF THE INVENTION

1. Field of the Invention

This invention relates to the metathesis (disproportionation) of olefins. More particularly, it relates to a process for carrying out a metathesis reaction in a fluidized bed reactor using a finely divided catalyst that promotes the metathesis reaction.

2. Description of the Prior Art

The catalyzed metathesis of olefins was first disclosed in 1964, and, because of its versatility, has since developed into a whole new field of its own within the universe of hydrocarbon chemistry.

Basically, the metathesis process utilizes a double bond displacement mechanism that involves the breaking and reformation of olefinic bonds, the type and number of bonds remaining unchanged. Starting with two different olefinic molecules, the reaction causes the displacement of double bond groups from each molecule to produce two new olefinic molecules that are not the same as the starting molecules. Displacement cleavage occurs at a double bond on each starting olefin molecule, and different olefin molecules are formed that have double bonds where the old double bonds were cleaved. For example, propylene is currently commercially produced by metathesizing 2-butene with an excess of ethylene. In this particular process, the double bonds in a molecule of 2-butene are cleaved as are the double bonds in a molecule of ethylene, and the resulting radicals reform to produce two new molecules of propylene. The process can be promoted with either homogeneous or heterogeneous catalyst systems comprised of one or more functional catalysts.

The metathesis of olefins is well understood and is fully and completely disclosed in U.S. Pat. No. 6,872,862 to Bridges, Powers, and Coleman.

Heretofore, metathesis reactions such as the propylene production process discussed above have been carried out using a fixed bed of catalyst through which flows the fluid (gas and/or liquid) olefin reactants, see U.S. Pat. Nos. 5,026,936 and 6,872,862. The catalyst employed in these fixed beds is a solid particle, typically pellet size, e.g., about 1/16 to ¼ inch in diameter and about 1/16 to ¼ inch in length.

Metathesis reactor (reactor) cycles between catalyst regeneration operations are often dictated by the pressure drop across the reactor. For example, the pressure drop across a reactor can climb steadily over the course of 2 to 4 weeks from an initial pressure of about 2 to 10 psig to a final pressure of over 30 psig. At this point in time in the operation of the reactor, the catalyst bed is sufficiently fouled to require shutdown of the process and a catalyst regeneration operation.

This pressure drop is usually caused by catalyst pellet attrition resulting in a buildup of catalyst fines in the reactor, or coke deposition on the catalyst pellets, or both. As the catalyst ages, accumulated catalyst fines in the catalyst bed increase not only the initial pressure drop across the bed, but also the rate of increase of the pressure drop over the period of time the bed is in operation.

Accordingly, it is desirable to have a metathesis process that is not subject to the vagaries of catalyst attrition and coke deposition in the catalyst bed.

### SUMMARY OF THE INVENTION

Pursuant to this invention a metathesis process is provided that employs a fluidized bed metathesis reactor and a stream of finely divided metathesis catalyst flowing through that reactor.

### BRIEF DESCRIPTION OF THE DRAWINGS

FIG. 1 shows a simplified flow sheet of a prior art metathesis process using a fixed bed of catalyst.

FIG. 2 shows a flow sheet of one embodiment within the process of this invention using a fluidized bed reactor.

### DETAILED DESCRIPTION OF THE INVENTION

For sake of clarity and brevity, this invention will be described in respect of the metathesis of 2-butene with ethylene to form propylene, but this invention is not so limited in its scope.

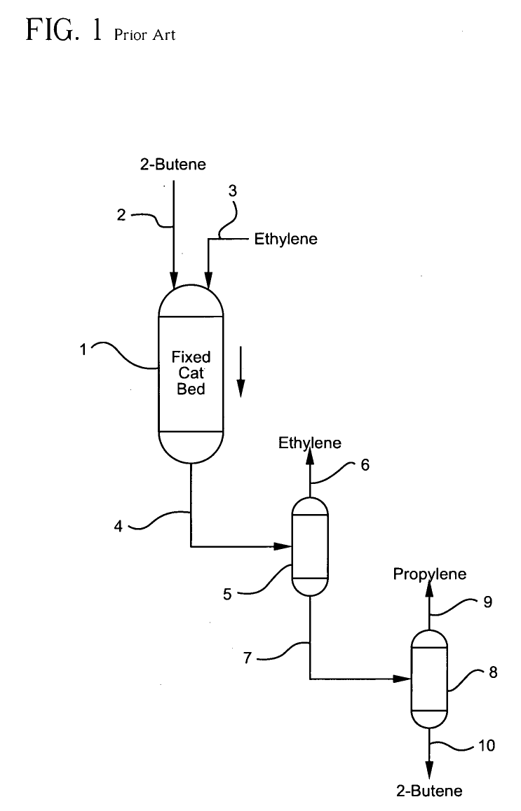

FIG. 1 shows a fixed bed of catalyst 1 into which flows reactant stream 2 composed of 2-butene and reactant stream 3 composed of ethylene. Catalyst bed 1 is maintained at operating conditions that favor, in the presence of the catalyst, the cleavage of double bonds in both the ethylene and 2-butene and the reformation of the resulting radicals into the desired propylene product.

The reaction mixture containing unreacted ethylene and 2-butene feeds and propylene product is passed by way of line 4 to a distillation column 5 that separates ethylene 6 as overhead from the reaction mixture for recycle to bed 1, if desired.

Bottoms stream 7 of column 5 is composed primarily of 2-butene and propylene. This mixture is separated into propylene product stream 9 and separate bottoms stream 10. Stream 10, composed essentially of 2-butene, can also be recycled to bed 1, if desired.

It is in bed 1 that attrited catalyst fines and/or coke can collect and drive the pressure drop across bed 1 (from inlets 2 and 3 to outlet 4) up to a level that requires the metathesis process to be terminated, and the catalyst in bed 1 regenerated.

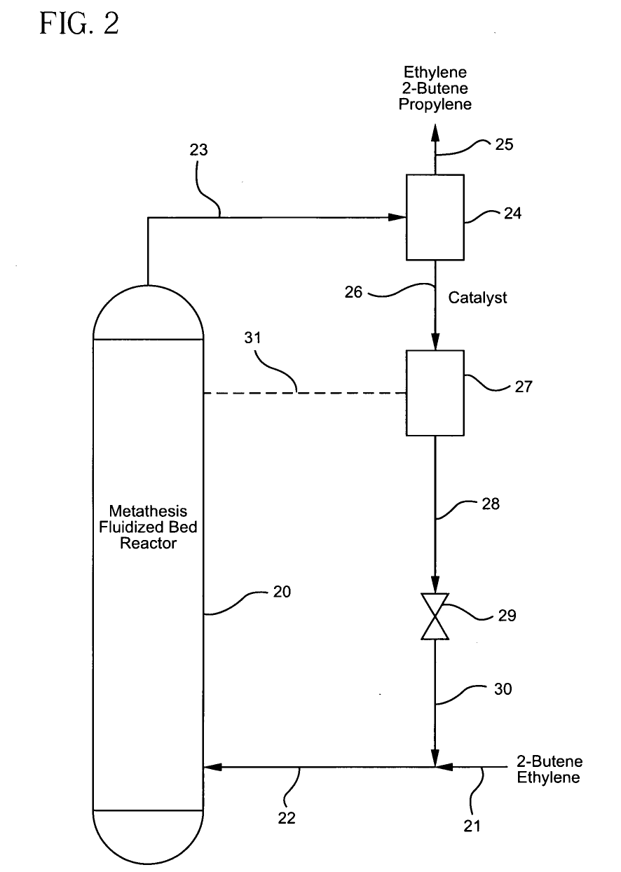

FIG. 2 shows one flow scheme within this invention. In this Figure a fluidized bed reactor 20 receives by way of conduit 22 a mixture of ethylene and 2-butene reactants from conduit 21 and finely divided (powdered) metathesis promoting catalyst from conduit 30. The mixture of reactants and catalyst flows into the bottom of reactor 20 and passes upwardly through the length of that reactor. The reactor is maintained at operating conditions that favor the conversion of one mole of ethylene and one mole of 2-butene to two moles of propylene.

The mixture of propylene product and unreacted ethylene and 2-butene is removed by way of line 23 and passed to a separator 24 wherein the fluid reaction mixture and product is separated from the solid catalyst powder. Unit 24 can be any conventional fluid/solid separator such as a cyclone or other centrifugal separator well known in the art. The reaction mixture is removed via line 25 for further processing, e.g., processing in columns 5 and 8 of FIG. 1, to recover the desired propylene product.

The catalyst that has been separated from the reaction mixture in unit 24 is removed from that unit by way of pipe 26, and passed into a catalyst storage hopper 27 wherein the catalyst resides until it is removed via conduit 28 for re-use in reactor 20. A metering valve 29 bleeds the desired amount of catalyst into the reactant mixture flowing in line 21 to form a new reactant/catalyst mixture to be passed into line 22 and reactor 20. If desired, heavier catalyst particles, if any, can be removed from reactor 20 and passed directly to hopper 27 as shown by dotted line 31. Catalyst can also, if desired, be separated and regenerated, e.g., with an air burn, and then returned to hopper 27.

The process of FIG. 2 addresses the problems of catalyst attrition and/or catalyst coking causing unacceptable pressure drops across the reactor, thereby allowing reactor 20 to operate continuously, and a substantially longer time, even years longer, between reactor shutdowns.

The process of this invention also allows for almost infinite flexibility for varying the make-up of the reactant/catalyst mixture that is to be subjected to metathesis conditions in reactor 20. For example, a 2-butene reactant stream may not be wholly 2-butene. It may contain minor amounts of 1-butene, and the amount of 1-butene contained in a reactant stream can vary over time. As reactant stream compositions change over time of operation, e.g., the 1-butene content in a 2-butene stream varies, by the process of this invention, the amount of ethylene and/or catalyst mixed with the 2-butene stream can be changed to accommodate the varying amount of 1-butene present. For example, if the 2-butene reactant contains varying amounts of 1-butene, and one of the catalyst components has olefin isomerization functionality (i.e., magnesium oxide), the magnesium oxide level in the catalyst passed to reactor 20 can be increased in any amount desired as the 1-butene content in the feed increases. Similarly, if the 1-butene content decreases, a matching decrease in magnesium oxide content can, with this invention, easily be affected. Thus, by this invention superior flexibility in operation is possible since the catalyst composition can be tailored to meet varying compositions of the reactant mix 21, and carry out a more efficient process.

The two or more reactants that form mix 21 can vary widely so long as they are olefins, with alpha or internal un-saturation. Generally, they can be mono-olefins having from 2 to 8 carbon atoms per molecule (C2 to C8 olefins). The reactants can be in the gaseous or liquid form or a combination thereof.

Suitable metathesis promoting catalysts include at least one of halides, oxides and/or carbonyls of molybdenum, tungsten, rhenium, and/or magnesium carried on a support, preferably an oxide support such as silica, alumina, titania, zirconia and mixtures thereof. Activating agents can also be included in the catalyst make-up. Such agents can include organo-metallic compounds such as tetra methyl tin; oxides such as alkaline earth metal oxides, alumina, silica, and mixtures thereof. Pursuant to this invention the catalyst or catalyst combinations employed will be finely subdivided to a solid particle range of from about 1 to about 300 microns.

The operating conditions maintained in reactor 20 can vary widely, but will generally be a temperature of from about 300 to about 800 degrees Fahrenheit (F), and a pressure of from about 200 to about 600 psig.

Reactor 20 can be a conventional fluidized bed reactor known in the art. This type of reactor includes the well known riser reactor and the ebullated bed reactor. In the operation of fluidized bed reactors pursuant to this invention, solid, particulate catalyst particles as defined above are made to behave as a fluid by the forced introduction of pressurized feed mixture 21 into the flow of catalyst particles from pipe 30. This mixing process causes the reactant/catalyst stream in pipe 22 to have many properties and characteristics of normal fluids, e.g., free-flow under the force of gravity and pumpability using conventional fluid transfer techniques. The overall result is fluidization of the combination of the reactants and solid powdery catalyst as it passes through conduit 22, reactor 20, and pipe 22. Metathesis occurs while the mixture of reactants and catalyst passes through the inside of reactor 20 and subjected to metathesis favoring operating conditions. As stated above, the reaction conditions can vary widely depending on the particular reactants and catalyst system used, so the dimensions of the reactor will also vary widely. However, reactor 20 will generally have a vertical height of from about 1 to about 100 feet thereby providing a reactant residence time inside the reactor itself of from about 10 milliseconds to about 10 minutes.

An ebullating bed reactor is particularly useful in this invention. This type of fluidized bed reactor completely fluidizes the solid catalyst powder particles and completely mixes these particles with the reactants present. In the ebullated bed process the reactant fluids and solid catalyst particles pass upwardly through reactor 20 at a rate such that the particles are forced into random motion as the mixture passes through that reactor. The formation of a catalyst bed that is in motion inside reactor 20 is controlled by a recycle fluid flow (not shown) so that at steady state, the bulk of the catalyst does not rise above a definable level in the reactor. Fluid reactants flow through the fluidized bed of catalyst into a more catalyst free zone in the upper portion of the reactor, and are removed from the upper end of the reactor at conduit 23. Catalyst carried over in line 23 with the removed reactants and product is separated, as in unit 24, for retention in hopper 27, and eventual reuse in reactor 20. Ebullating bed reactors and their process of operation are fully disclosed in U.S. Pat. No. 5,494,570.

### EXAMPLE

A mixture of about 16 weight percent (wt %) 1-butene and about 84 wt % 2-butene together with a molar excess ethylene is mixed with a powdered catalyst system composed of tungsten oxide and magnesium oxide. The catalyst system is composed of finely divided particles ranging from about 1 to about 100 microns in their largest cross-sectional dimension. This mixture of reactants and catalyst is introduced into an operating ebullating bed reactor that is maintained at a temperature of about 600F and a pressure of about 350 psig. The ebullated bed is operated at a flow rate that provides a residence time for the reactants in the reactor of about 10 minutes.

A mixture of propylene, unreacted ethylene, unreacted 2-butene, 1-butene, and propylene is recovered overhead from the reactor, and the propylene separated therefrom as a product of the process.

We claim:

1. In a method for metathesizing at least two olefins wherein said olefins are contacted with a metathesis promoting solid catalyst under operating conditions that favor the transformation of said olefins to at least one other olefin as the product of the process, the improvement comprising carrying out said metathesis in a fluidized bed reactor using finely subdivided metathesis catalyst.

2. The method of claim 1 wherein said olefins are selected from the group consisting of C2 to C8 olefins having at least one of alpha or internal double bonds.

3. The method of claim 1 wherein said finely subdivided catalyst has a particle size of from about 1 to about 300 microns.

4. The method of claim 1 wherein said fluidized bed reactor is one of a riser reactor and an ebullated bed reactor.

5. The method of claim 1 wherein said catalyst is at least one of halides, oxides, and carbonyls of at least one of molybdenum, tungsten, rhenium, and magnesium carried on a solid support.

6. The method of claim 5 wherein said catalyst contains at least one activating agent.

7. The method of claim 1 wherein said operating conditions are a temperature of from about 300 to about 800F, and a pressure of from about 200 to about 600 psig.

8. The method of claim 1 wherein said olefins are at least ethylene and 2-butene, said catalyst is magnesium oxide and tungsten oxide supported on silica, and said at least one other olefin product is propylene.
